(19) United States
(12) Patent Application Publication
(10) Pub. No.: US 2026/0151057 A1
(43) Pub. Date: Jun. 4, 2026

(54) NON-INVASIVE HEALTH MONITORING DEVICE

(71) Applicant: Maximillian Byron Kopp, Blue Bell, PA (US)

(72) Inventor: Maximillian Byron Kopp, Blue Bell, PA (US)

(21) Appl. No.: 19/203,391

(22) Filed: May 9, 2025

Related U.S. Application Data
(60) Provisional application No. 63/726,463, filed on Nov. 29, 2024.

(51) Int. Cl. A61B 5/1455; A61B 5/00; A61B 5/145

(52) U.S. Cl. CPC A61B 5/1455; A61B 5/0004; A61B 5/14532; A61B 5/681; A61B 5/702; A61B 5/7264; A61B 5/742; A61B 2562/0238

(57) ABSTRACT

A non-invasive health monitoring device includes a housing configured to receive a body part of a user, a light emitter disposed within the housing and configured to emit linearly polarized light, and a polarization modulator comprising a germanium-selenium (GeSe) crystal arranged in an optical path of the light emitter. The polarization modulator modulates a polarization state of light transmitted through the body part of the user. A photodetector disposed within the housing receives the modulated light and detects changes in the polarization state. A processor communicatively coupled to the photodetector analyzes the polarization changes relative to the emitted linearly polarized light and determines a concentration of a target analyte within the body part. A corresponding measurement indicative of the target analyte concentration is output by the processor.

### TECHNICAL FIELD

The present disclosure generally relates to non-invasive health monitoring devices, and more specifically, to a non-invasive health monitoring device comprising a polarization modulator with a germanium-selenium (GeSe) crystal for determining a target analyte concentration within a body part of a user.

### BACKGROUND

Non-invasive health monitoring devices have emerged as essential tools in personalized healthcare, offering convenience, safety and continuous access to physiological data without the need for puncturing skin or extracting blood. Such devices address a broad spectrum of health monitoring applications including blood glucose tracking, oxygen saturation measurement, and monitoring of other vital signs. Recent advances in optical and photonic technologies have enabled devices to perform sensing based on light interaction with human tissue, providing new pathways for continuous health diagnostics. However, despite substantial progress, technical limitations persist in enhancing accuracy, achieving high signal-to-noise ratio, and enabling reliable analyte-specific detection through compact, wearable platforms.

U.S. Pat. No. 12,076,136 describes a method for monitoring a subject using a clinical-grade glucose monitor to calibrate non-invasive sensors worn on the body. The system employs one or more light emitting diodes at different wavelengths and photodetectors to measure glucose-related optical properties of the skin. However, the method requires initial calibration with invasive glucose monitors and complex multi-sensor arrays, thereby limiting its utility as a stand-alone non-invasive solution and complicating its deployment in wearable or compact formats without laboratory-grade support.

U.S. Pat. No. 9,974,451 discloses a health care band configured to attach a biosensor to a patient, the biosensor comprising multiple sensors for collecting vital signs and wirelessly communicating the data to an electronic medical record (EMR) network. While this approach enables remote health data collection and real-time data association with patient profiles, the device primarily focuses on network integration and lacks advanced sensing modalities to detect biochemical concentrations, such as blood glucose.

U.S. Pat. No. 9,649,055 presents a wearable monitoring device including a flexible substrate with multiple radiation sources and detectors arranged in a defined geometry. The device is configured to dynamically adjust its operational geometry by varying source intensity and detector gain to optimize quality metrics for monitoring physiological parameters. While the device improves adaptability in wearable settings and enhances signal quality using dynamic control of optical components, the disclosed system focuses solely on geometric and electrical parameters rather than polarization-state-driven signal enhancement.

US Patent App. Pub. 20210137466 discloses a user equipment configured to interface with an external biosensor for physiological monitoring. The device comprises a display, a memory, and at least one processing circuit programmed to receive and process biosensor data, including photoplethysmographic (PPG) signals at multiple wavelengths, to determine nitric oxide (NO) levels in blood flow. The system further includes a graphical user interface (GUI) configured to present the received biosensor data to a user. While the invention enables user-friendly display and interpretation of biosensor-derived health information, it is primarily focused on signal processing and user interaction layers, and does not address improvements to sensor architecture, structural design, or physical mechanisms to enhance biochemical analyte detection accuracy.

Other polarization based optical sensors—such as those using liquid crystal modulators or LiNbO₃ waveplates—require complex multi element assemblies and external calibration routines. These bulky systems are unsuitable for compact, standalone wearables that must operate reliably without laboratory grade support.

Thus, a technical challenge remains in developing a non-invasive health monitoring device that provides analyte-specific measurements using a simple, compact and self-contained optical system capable of standalone operation. Devices relying on multiple sensors, wavelength multiplexing, or external calibration routines increase system complexity, size and power demands, making them unsuitable for continuous personal monitoring or discreet wearable configurations. Furthermore, achieving high analyte specificity without blood extraction remains a core challenge due to overlapping tissue signals, scattering losses and limited sensitivity of conventional sensors.

Accordingly, there remains a need for a self-contained, miniaturized polarization modulator that (1) operates in the near-infrared spectrum, (2) delivers high birefringence sensitivity for analyte detection, and (3) is compatible with flexible, low-cost manufacturing processes. Such a system should accurately detect target analyte concentrations without relying on invasive procedures or external calibration with clinical-grade monitors. Furthermore, the device should support both real-time and on-demand measurements, thereby offering a versatile, analyte-specific, and user-friendly solution for non-invasive health monitoring.

### SUMMARY

The present disclosure provides a non-invasive health monitoring device comprising a housing configured to receive a body part of a user, a light emitter disposed within the housing, the light emitter configured to emit linearly polarized light and the emitted linearly polarized light transmitted through the body part of the user, a polarization modulator disposed within the housing on an optical path of the light emitter, the polarization modulator comprising a germanium-selenium (GeSe) crystal configured to modulate a polarization state of transmitted light after interaction of the emitted linearly polarized light with the body part of the user, a photodetector disposed within the housing on an optical path of the polarization modulator, the photodetector configured to receive modulated light after modulation of the light by the polarization modulator and detect a polarization state of the modulated light, and a processor communicatively coupled to the photodetector, the processor configured to analyze changes in the polarization state of the modulated light with respect to a polarization state of the emitted linearly polarized light to determine a target analyte concentration within the body part of the user and output a corresponding measurement indicative of the target analyte concentration. This configuration enables accurate determination of target analyte concentrations in a body part of the user using changes in polarization states of light without requiring invasive techniques.

In one embodiment, the non-invasive health monitoring device is a glucometer and the target analyte concentration is blood glucose concentration. This enables measurement of blood glucose concentration without invasive procedures. Although this embodiment focuses on blood glucose, the same polarization-modulation approach applies to other chiral analytes—such as lactate or cholesterol—that induce optical rotation in biological tissue.

In another embodiment, the non-invasive health monitoring device further comprises an optical sensing chamber, and is implemented as a finger-insert device configured to receive a finger of the user into the optical sensing chamber for monitoring the target analyte concentration on demand. As used herein, ‘optical sensing chamber’ means any internal cavity or aperture in the device housing configured to receive a user's body part and confine light propagation through the tissue. This enables on-demand target analyte monitoring via a compact finger-insert implementation.

In yet another embodiment, the non-invasive health monitoring device is implemented as a wrist-wearable device configured to receive a wrist of the user for continuous real-time monitoring of the target analyte concentration. This provides continuous real-time monitoring in a wearable format.

In a further embodiment, the processor is configured to execute an artificial intelligence (AI) algorithm for processing variations in the polarization states of the modulated light to improve accuracy of measurement of target analyte concentrations. This enhances the accuracy of target analyte measurement using AI-based processing of polarization variations.

In still another embodiment, the GeSe crystal is processed using mechanical exfoliation to achieve a uniform nanoscale thickness. This improves the quality of polarization modulation through the GeSe crystal by ensuring nanoscale consistency.

In a further embodiment, the GeSe crystal is subjected to thermal treatment at temperatures between 300° C. and 500° C. to enhance birefringence properties of the GeSe crystal. This improves the polarization modulation performance of the GeSe crystal.

In another embodiment, a surface passivation layer is deposited on the GeSe crystal to prevent oxidation and degradation of the GeSe crystal. This increases the operational stability and longevity of the GeSe crystal under real-world conditions.

In yet another embodiment, the non-invasive health monitoring device further comprises an integrated display to present the measurement indicative of the target analyte concentration. This allows users to directly view results on the device itself.

In still another embodiment, the non-invasive health monitoring device further comprises a wireless communication module connected to the processor, the wireless communication module configured to transmit the measurement indicative of the target analyte concentration to an external computing device. This enables remote access and integration of measurement data into external systems.

It is to be appreciated that all the aforementioned embodiments can be combined. All steps which are performed by the various entities described in the present disclosure as well as the functionalities described to be performed by the various entities are intended to mean that the respective entity is adapted to or configured to perform the respective steps and functionalities. It will be appreciated that features of the present disclosure are susceptible to being combined in various combinations without departing from the scope of the present disclosure as defined by the appended claims. Additional aspects, advantages, features, and objects of the present disclosure would be made apparent from the drawings and the detailed description of the illustrative implementations construed in conjunction with the appended claims that follow.

The present disclosure also provides a method for non-invasive analyte measurement, the comprising emitting linearly polarized light, transmitting the light through a body part, modulating the light via a GeSe crystal, detecting a polarization state change in the modulated light and determining a target analyte concentration based on the detected change.

Additionally, the present disclosure provides a non-transitory computer-readable medium storing instructions that, when executed by a processor, cause the processor to perform the aforementioned method.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Notwithstanding any other forms which may fall within the scope of the present invention, preferred embodiments of the disclosure will now be described, by way of example only, with reference to the accompanying drawings in which: to the drawings, in which:

FIG. 1 illustrates an exploded view of a non-invasive health monitoring device, in accordance with an embodiment of the present disclosure.

FIG. 2 illustrates another exploded view of the non-invasive health monitoring device of FIG. 1, in accordance with an embodiment of the present disclosure.

FIG. 3 illustrates another exploded view of the non-invasive health monitoring device of FIGS. 1-2, in accordance with an embodiment of the present disclosure.

FIG. 4 illustrates a front perspective view of a non-invasive health monitoring device (such as the non-invasive health monitoring device of FIGS. 1-3), in accordance with an embodiment of the present disclosure.

FIG. 5 illustrates a front perspective view of the non-invasive health monitoring device of FIG. 4, in accordance with an embodiment of the present disclosure.

FIG. 6 illustrates a front view of an upper-arm wearable device, in accordance with an embodiment of the present disclosure.

FIG. 7 shows a process of mechanical exfoliation, in accordance with an embodiment of the present disclosure.

FIG. 8 shows the GeSe crystal of FIG. 7 subjected to thermal treatment, in accordance with an embodiment of the present disclosure.

FIG. 9 shows the polarization modulator (such as the polarization modulator of FIG. 1) ready for testing, in accordance with an embodiment of the present disclosure.

FIG. 10 shows the tested polarization modulator of FIG. 9 integrated into a processor (such as the processor of FIGS. 1-3) to obtain an integrated circuit (IC), in accordance with an embodiment of the present disclosure.

FIG. 11 shows an implementation of the non-invasive health monitoring device of FIG. 1 with a companion app for a smartphone, in accordance with an embodiment of the present disclosure.

FIG. 12 shows a three-dimensional view of an upper-arm wearable armband for the non-invasive health monitoring device, in accordance with an embodiment of the present disclosure.

### DETAILED DESCRIPTION OF THE EMBODIMENTS

The example embodiments herein and the various features and advantageous details thereof are explained more fully with reference to the non-limiting embodiments that are illustrated in the accompanying drawings and detailed in the following description. Descriptions of well-known components and processing techniques are omitted to not unnecessarily obscure the embodiments herein. The description herein is intended merely to facilitate an understanding of ways in which the example embodiments herein can be practiced and to further enable those of skill in the art to practice the example embodiments herein. Accordingly, this disclosure should not be construed as limiting the scope of the example embodiments herein.

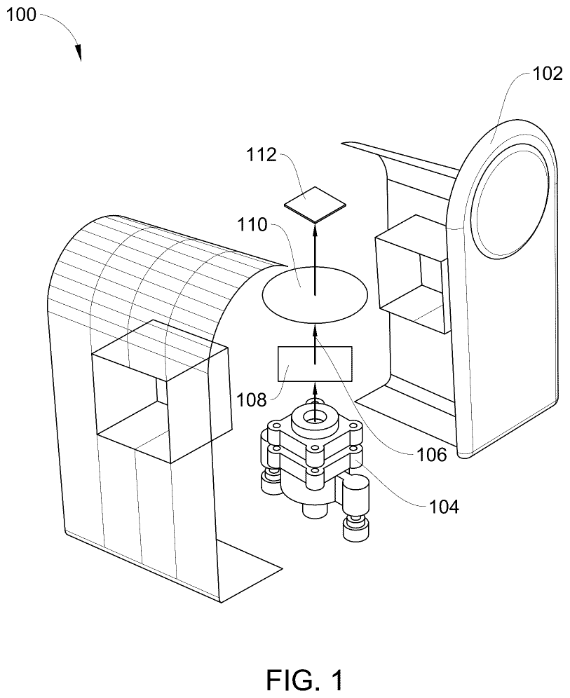

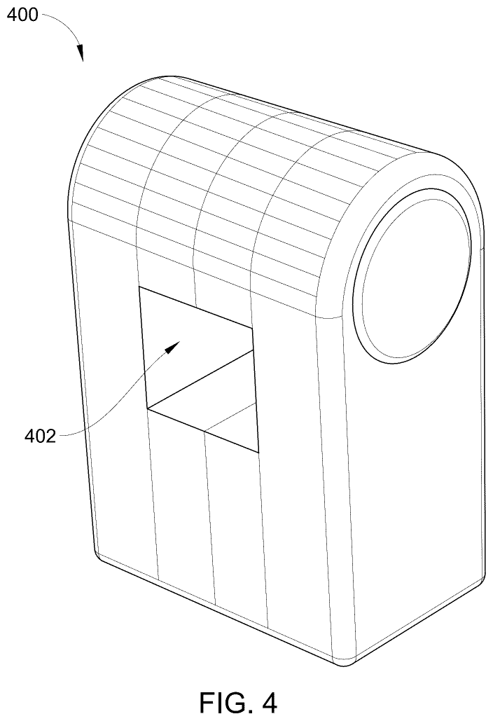

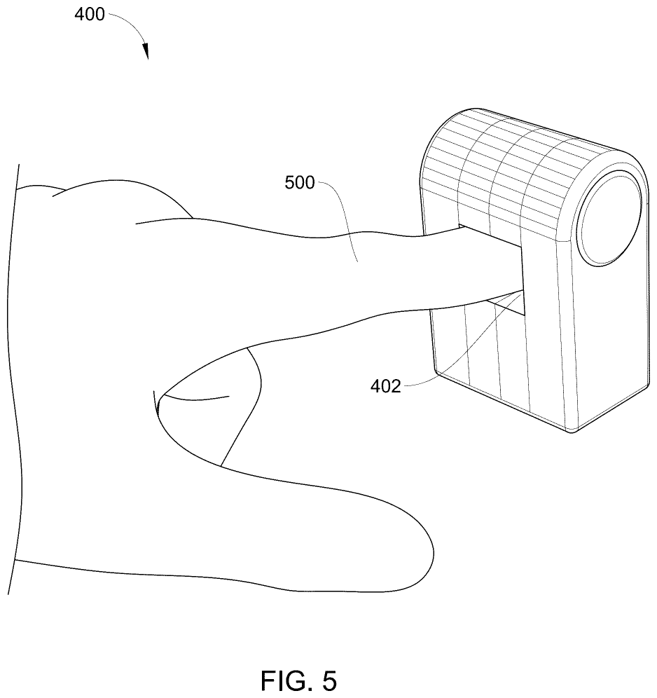

Referring to FIG. 1, there is shown an exploded view of a non-invasive health monitoring device 100, in accordance with an embodiment of the present disclosure. The device 100 comprises a housing 102 configured to receive a body part of a user and a light emitter 104 disposed within the housing 102. The light emitter 104 is configured to emit linearly polarized light 106. The emitted linearly polarized light 106 is transmitted through the body part of the user. The term “housing” as used throughout the disclosure relates to a structural enclosure that physically supports and protects internal optical and electronic components of the device 100. The housing 102 is configured to interface with a body part of a user, such as by receiving a finger or coming into contact with a wrist of the user and may therefore define an internal cavity or optical sensing chamber (shown in FIGS. 4-5) for positioning the body part along an optical path of the light emitter 104. The term “light emitter” as used throughout the disclosure relates to an active optical component that produces and emits linearly polarized light 106 for non-invasive interaction with a biological tissue of the body part of the user received within the housing 102.

The housing 102 may be implemented in ergonomic or contoured geometries to conform to anatomical surfaces and may be manufactured from biocompatible materials such as medical-grade polymers such as, polycarbonate, acrylonitrile butadiene styrene, lightweight metals, or silicone-based composites. The housing 102 facilitates accurate placement and alignment of the body part of the user relative to the light emitter 104 to ensure effective optical transmission of the linearly polarized light 106 through the biological tissue. The term “linearly polarized light” as used throughout the disclosure relates to light whose electric field oscillates in a single plane, which enhances the sensitivity of optical detection systems to birefringence or polarization state changes induced by interaction with target analytes present within biological tissue of the body part of the user. The housing 102 provides physical protection of the light emitter 104 and downstream optical components, environmental sealing against dust and moisture, and mechanical stabilization of a user interface (such as the optical sensing chamber) to reduce motion artifacts during measurement. The implementation of various optoelectronic components of the non-invasive health monitoring device 100, such as the light emitter 104, within the housing 102, enables controlled delivery and confinement of the optical path of light within a protective structure of the housing 102, thereby minimizing environmental interference and mechanical misalignment during operation.

The light emitter 104 may be implemented using one or more light sources such as laser diodes, vertical-cavity surface-emitting lasers (VCSELs), or light-emitting diodes (LEDs), integrated with polarizing optical elements such as, polarizing films, polarizing beam splitters, or polarization gratings. The light emitter 104 is configured to generate linearly polarized light 106 of a specific wavelength or range of wavelengths optimized for optical penetration and biochemical specificity when transmitted through the biological tissue of the body part of the user. The inclusion of the light emitter 104 enables wavelength-specific interaction with target analytes to be detected within the body part of the user, improving optical signal quality, and reducing noise caused by diffuse scattering in the biological tissue.

The linearly polarized light 106 propagates through the body part, allowing subsequent optoelectronic components to detect variations in polarization caused by interaction with glucose molecules or other target analytes. The generation and use of linearly polarized light 106 increases signal-to-noise ratio in polarization-sensitive detection, enhanced selectivity toward analyte-induced optical effects, and improved measurement repeatability under varying conditions of the biological tissue of the user. In one example, the alignment of the light emitter 104 within the housing 102 is selected such that the emitted linearly polarized light 106 enters the tissue at a predefined angle, optimizing interaction with subsurface capillary beds. The emitted linearly polarized light 106 serves as the initiating optical signal for downstream polarization modulation and detection stages, forming a critical component of the non-invasive analyte monitoring system. This alignment maximizes analyte-specific optical interaction within the most informative layers of the biological tissue, thereby improving the accuracy and depth sensitivity of the system 100.

The system 100 further comprises a polarization modulator 108 disposed within the housing 102 on an optical path of the light emitter 104. The polarization modulator 108 comprises a germanium-selenium (GeSe) crystal (or other layered 2D birefringent materials, e.g., black phosphorus) configured to modulate a polarization state of transmitted light after interaction of the emitted linearly polarized light with the body part of the user. The term “polarization modulator” as used throughout the disclosure relates to an optoelectronic component configured to modify the polarization state of light based on interaction with optically active or birefringent media. The polarization modulator 108 is disposed downstream of the body part in the optical path of the emitted linearly polarized light 106 and is configured to receive light that has passed through the biological tissue. The transmitted light carries polarization state information altered by interactions with target analytes such as glucose. The placement of the polarization modulator 108 within the housing 102 enables precise alignment with other optoelectronic components, reduces optical path contamination by environmental interference, and ensures repeatable modulation of polarization states under compact, portable, and field-deployable operating conditions.

The polarization modulator 108 comprises a germanium-selenium (GeSe) crystal, selected for electro-optic and birefringent properties thereof. The GeSe crystal modulates the polarization state of the transmitted light based on the optical activity of the biological tissue and the presence of analytes that induce a measurable change in polarization angle. The polarization modulator 108 is configured to transform the polarization state of the transmitted light into a modified polarization state suitable for differential analysis by downstream optoelectronic components. The use of the GeSe crystal enables high modulation depth, low insertion loss, and spectral compatibility with near-infrared wavelengths relevant to interrogation of the biological tissue. The inherent material anisotropy of GeSe facilitates robust detection of subtle polarization rotations induced by target analytes present in low-concentrations.

The inclusion of the GeSe crystal in the polarization modulator 108 enables sensitive detection of optical rotation or polarization modulation induced by glucose or other target analytes present in interstitial fluid. The GeSe crystal may be implemented in one or more fixed or rotatable orientations and may be driven by mechanical, thermal or electric field-based actuation systems depending on the sensing configuration. The action performed by the polarization modulator 108 is critical for enhancing the signal-to-noise ratio in analyte-specific detection and improving the accuracy of non-invasive measurements in dynamic physiological environments. Additionally, the polarization modulator 108 enables adaptability to real-time modulation control, tunability of phase retardation characteristics, and compatibility with compact, low-power optical sensing architectures.

Moreover, the system 100 comprises a photodetector 110 disposed within the housing 102 on an optical path of the polarization modulator 108. The photodetector 110 is configured to receive modulated light after modulation of the light by the polarization modulator 108. The photodetector 110 is configured to detect a polarization state of the modulated light. The term “photodetector” as used throughout the disclosure relates to an optical sensing element configured to convert incident light into electrical signals for quantitative analysis. The photodetector 110 is positioned to capture light whose polarization characteristics have been altered based on analyte-specific interactions with biological tissue and subsequently modulated by the polarization modulator 108. In one example, the photodetector 110 comprises one or more polarization-resolving photodiodes, photodiode arrays, or charge-coupled devices (CCDs), optionally in combination with polarization filters, beam splitters, or polarization gratings. The photodetector 110 may be configured to discriminate between different polarization angles or intensities of the received modulated light to enable extraction of analyte-specific signals.

The photodetector 110 is associated with high sensitivity to polarization-dependent optical variations, compatibility with real-time signal acquisition, and robustness against ambient light interference due to polarization selectivity. The arrangement of the photodetector 110 within the housing 102 facilitates low-loss optical coupling from the polarization modulator 108, minimizes alignment errors, and supports integration within a compact sensing module. Additionally, the photodetector 110 enables precise measurement of optical rotation or retardation resulting from glucose-induced birefringence effects, thereby contributing to the overall accuracy and reliability of non-invasive analyte monitoring.

The system 100 also comprises a processor 112 communicatively coupled to the photodetector 110. The processor 112 is configured to analyze changes in the polarization state of the modulated light with respect to a polarization state of the emitted linearly polarized light 106 to determine a target analyte concentration within the body part of the user and output a corresponding measurement indicative of the target analyte concentration. The term “processor” as used throughout the disclosure relates to an electronic computing unit configured to perform signal analysis, data processing, and control operations within the system 100. The processor 112 is implemented as a microcontroller, application-specific integrated circuit (ASIC), field-programmable gate array (FPGA), or digital signal processor (DSP), and includes one or more memory modules storing executable instructions. The processor 112 is operatively connected to the photodetector 110 to receive signals representative of the polarization state of the modulated light, and is further configured to compare the signals with reference data representing the polarization state of the emitted linearly polarized light 106.

The processor 112 enables extraction of meaningful polarization-based features associated with the presence and concentration of one or more target analytes such as glucose. The processor 112 evaluates polarization shifts or angular deviations introduced during tissue interaction and modulation, allowing determination of analyte-specific optical signatures. The processor 112 provides real-time or near-real-time computation of concentration values, and facilitates display, storage, or wireless transmission of the resulting measurement output. The integration of the processor 112 within the device 100 supports closed-loop data analysis, device calibration, and adaptive signal processing for enhanced accuracy, repeatability, and resilience to environmental or physiological variability.

In one embodiment, the non-invasive health monitoring 100 is a glucometer. The target analyte concentration is blood glucose concentration. The term “glucometer” as used throughout the disclosure relates to a non-invasive diagnostic device configured to determine the concentration of glucose in blood or interstitial fluid of a user without requiring penetration of the skin or extraction of bodily fluids. When configured as a glucometer, the system 100 allows real-time monitoring of glucose levels by analyzing polarization changes in light passing through biological tissue of the body part of the user, such as a finger, wrist, earlobe and the like. The implementation of the non-invasive health monitoring 100 as the glucometer facilitates pain-free and convenient glucose tracking, thereby improving user compliance, especially in diabetic patients requiring frequent glucose assessments.

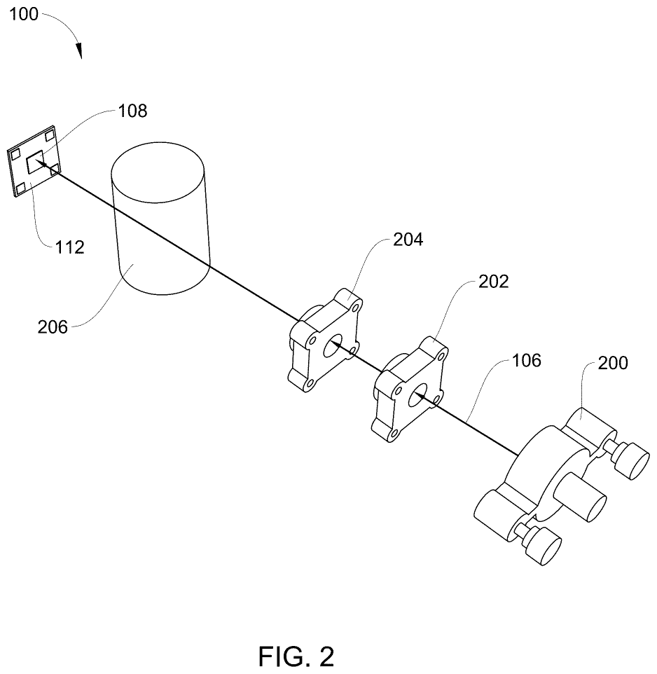

Referring now to FIG. 2, there is shown another exploded view of the non-invasive health monitoring device 100 of FIG. 1, in accordance with an embodiment of the present disclosure. The housing 102 has been omitted in FIG. 2. Further, the light emitter 104 has been has further broken down into a light source 200 configured to emit the linearly polarized light 106 and a pair of lenses 202, 204 configured to focus the linearly polarized light 106 emitted by the light source 200. The separation of the light emitter 104 into distinct components allows for optimized optical design and greater control over the emitted light properties. The inclusion of the pair of lenses 202, 204 provides precise beam shaping and collimation, ensuring that the linearly polarized light 106 is directed along the intended optical path with minimal divergence or loss. This focused delivery of light improves interaction with the tissue and enhances the quality of the polarization-modulated signals received by the photodetector 110.

The polarization modulator 108 is shown to be integrated into the processor 112. Further, an exemplary cylindrical test sample 206 is arranged in the optical path of the linearly polarized light 106 to illustrate the effects of the body part of the user on the linearly polarized light 106. The exemplary cylindrical test sample 206 serves to visually demonstrate how interaction with biological tissue alters the polarization state of the linearly polarized light 106. Integration of the polarization modulator 108 with the processor 112 allows efficient processing of polarization-modulated signals directly within the processor, eliminating the need for intermediate circuitry and reducing system complexity. This integration also supports the compact design of the device 100, enhancing its suitability for portable and wearable applications.

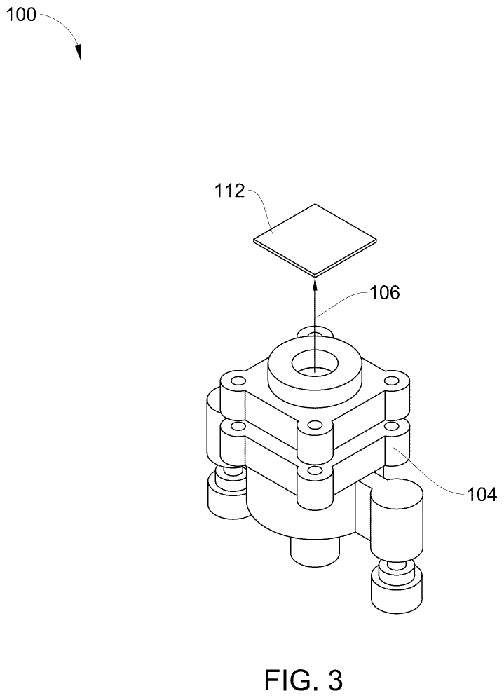

Referring now to FIG. 3, there is shown another exploded view of the non-invasive health monitoring device 100 of FIGS. 1-2, in accordance with an embodiment of the present disclosure. The housing 102, the polarization modular 108 and the photodetector 110 have been omitted from FIG. 3 to clearly indicate the emission of the linearly polarized light 106 from the light emitter 104. FIG. 3 highlights the direct optical path of the linearly polarized light 106, emphasizing the alignment and precision required for effective polarization-based sensing. The simplified representation provides insight into how the linearly polarized light 106 propagates through the non-invasive health monitoring device 100 and interacts with the tissue, facilitating accurate determination of the target analyte concentration.

Referring now to FIG. 4, there is shown a front perspective view of a non-invasive health monitoring device 400 (such as the non-invasive health monitoring device 100 of FIGS. 1-3), in accordance with an embodiment of the present disclosure. The non-invasive health monitoring device 400 comprises an optical sensing chamber 402. As shown, the non-invasive health monitoring device 400 is implemented as a finger-insert device configured to receive a finger (shown in FIG. 5) of the user into the optical sensing chamber 402 for monitoring the target analyte concentration on demand. The term “optical sensing chamber” as used throughout the disclosure relates to a defined internal region within the non-invasive health monitoring device 400 that houses optical components and provides an isolated optical pathway for interaction of light with the biological tissue of the body part of the user. The optical sensing chamber 402 is configured to receive the body part, such as the finger, such that incident light from the light emitter can interact with subdermal tissue and be collected after transmission or reflection. The optical sensing chamber 402 may be implemented using opaque, light-absorbing materials to minimize ambient light interference and improve measurement fidelity. In some implementations, the optical sensing chamber 402 may include internal baffling structures or coatings to suppress stray reflections and guide light along a controlled path.

When implemented as the finger-insert device, the non-invasive health monitoring device 400 allows consistent anatomical positioning of the finger with respect to the optical pathway, enabling reproducible optical measurements of target analyte concentration. The physical structure of the finger-insert form factor provides mechanical stability and minimizes motion artifacts during use, which is particularly beneficial during on-demand glucose monitoring scenarios. The optical sensing chamber 402 enables encapsulation and alignment of optical elements relative to the received finger, thereby facilitating high-quality signal acquisition and improving overall reliability of determination of the target analyte concentration.

Referring now to FIG. 5, there is shown a front perspective view of the non-invasive health monitoring device 400 of FIG. 4, in accordance with an embodiment of the present disclosure. As shown, a finger 500 is received into the optical sensing chamber 402 such that soft tissue regions containing vascular and interstitial structures are positioned along the optical path defined within the non-invasive health monitoring device 400. In this configuration, the inserted finger 500 enables the emitted linearly polarized light to interact with subdermal analyte-rich regions, including capillary beds and interstitial fluid compartments, thereby facilitating optical sensing of the target analyte concentration.

The insertion of the finger 500 into the optical sensing chamber 402 provides a consistent and anatomically repeatable sensing interface that minimizes measurement variability across multiple uses. The arrangement of the optical sensing chamber 402 to receive the finger 500 further ensures that transmitted and modulated light signals can be acquired under stable and reproducible conditions, improving detection precision and enhancing the accuracy of the analyte concentration estimation.

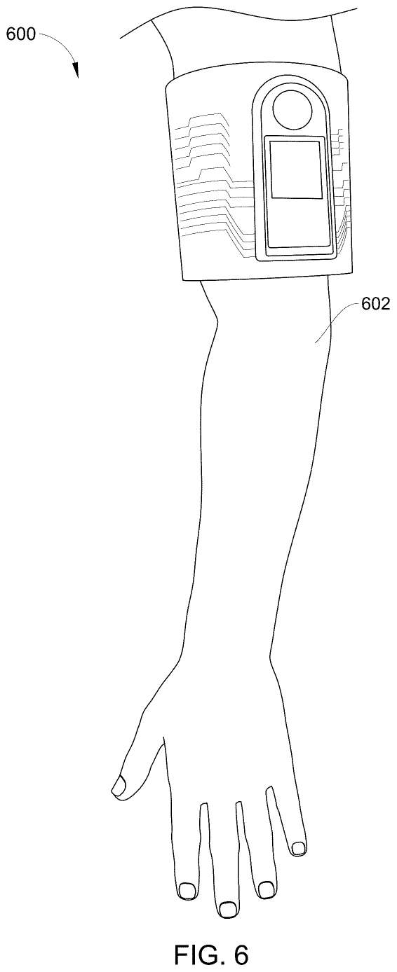

Referring to FIG. 6, there is shown a illustrates a front view of an upper-arm wearable device 600, in accordance with an embodiment of the present disclosure. As shown, the non-invasive health monitoring device 600 is implemented as a upper-arm configured to worn on an upper arm 602 of the user for continuous real-time monitoring of the target analyte concentration. An arm strap 604 of the non-invasive health monitoring device 600 is worn on the upper arm 602 of the user, positioned in alignment with the optical path defined within the upper arm wearable device 600, allowing emitted linearly polarized light to pass through vascularized biological tissue such as the radial or ulnar arteries. The implementation of the upper arm wearable device 600 as the upper-arm wearable device supports ongoing acquisition of polarization-modulated optical signals for uninterrupted tracking of the target analyte concentration over extended durations.

The upper-arm wearable form factor enables passive and user-independent operation, allowing the user to engage in daily activities without interrupting measurement. The continuous placement of the upper-arm 602 within the device 600 facilitates longitudinal monitoring of physiological fluctuations and trends, supporting time-resolved analysis of the target analyte concentrations and enabling proactive health interventions based on real-time data.

In the finger-insert embodiment (FIGS. 4-5), the device provides rapid, on-demand spot measurements of analyte concentration. In contrast, the upper-arm wearable embodiment (FIG. 6) supports continuous, longitudinal monitoring, enabling trend analysis without user intervention.

Referring back to the non-invasive health monitoring device shown in FIGS. 1-3, in one embodiment, the processor 112 is configured to execute an artificial intelligence (AI) algorithm for processing variations in the polarization states of the modulated light to improve accuracy of measurement of target analyte concentrations. The execution of the AI algorithm enables dynamic correction of signal artifacts, noise suppression, and compensation for inter-individual variability in tissue optical properties. The AI algorithm may be implemented using machine learning models trained on a dataset of polarization signatures correlated with reference analyte concentrations, allowing the processor 112 to infer glucose or other analyte levels with higher precision.

The learning of complex non-linear relationships between polarization changes and analyte concentrations enables the AI algorithm to provide enhanced discrimination of signal features that are otherwise obscured by physiological noise or environmental fluctuations. The use of the AI algorithm also facilitates real-time adaptation of detection parameters based on historical trends or observed inconsistencies, supporting robust and accurate operation across diverse users and conditions.

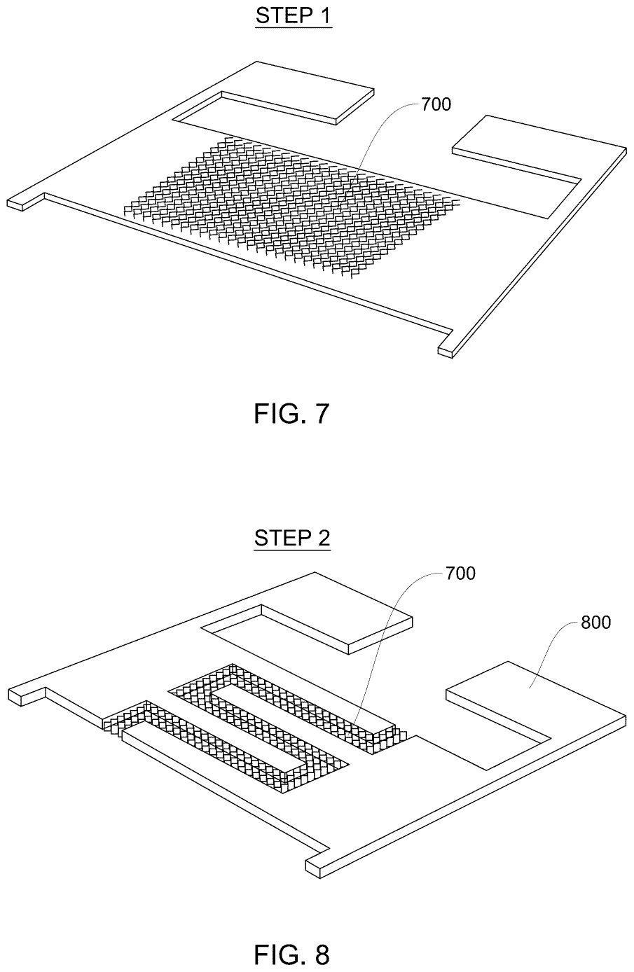

Referring to FIGS. 7-10, there is illustrated a successive technique of processing of the GeSe crystal using mechanical exfoliation to obtain an integrated chip 1000, in accordance with various embodiments of the present disclosure.

FIG. 7 shows a process of mechanical exfoliation, in accordance with an embodiment of the present disclosure. The mechanical exfoliation is Step 1 of a process and involves physically cleaving the layered GeSe material 700 to isolate ultrathin flakes with uniform optical properties across their surface and achieve a uniform nanoscale thickness. The uniform nanoscale thickness of the GeSe crystal allows precise modulation of the polarization state of transmitted light, improving consistency and reliability of polarization-based measurements across multiple uses. A thinner GeSe crystal also supports reduced optical path delay and enables compact integration within miniaturized sensing architectures, such as wearable or point-of-care devices. Additionally, exfoliated GeSe structures enhance interaction with transmitted light due to stronger confinement of the optical field, which increases the sensitivity of the polarization modulator 108 to analyte-induced changes in polarization.

FIG. 8 shows the GeSe crystal 700 of FIG. 7 subjected to thermal treatment, in accordance with an embodiment of the present disclosure. The thermal treatment is Step 2 of the process. The GeSe crystal is subjected to thermal treatment at temperatures between 300° C. and 500° C. to enhance birefringence properties of the GeSe crystal. The thermal treatment modifies the internal crystal lattice structure of the GeSe crystal, resulting in improved optical anisotropy and enhanced polarization modulation capability. The enhanced birefringence properties of the thermally treated GeSe crystal increase the polarization sensitivity of the polarization modulator 108 to subtle changes in optical rotation caused by the presence of target analytes within biological tissue. The improved birefringence enables greater resolution in detecting variations in polarization state, which in turn contributes to higher accuracy in non-invasive analyte concentration measurement. The thermal treatment also stabilizes the performance characteristics of the GeSe crystal 700 under fluctuating environmental or physiological conditions, allowing more consistent operation in wearable and continuous monitoring applications.

In yet another embodiment, a surface passivation layer 800 is deposited on the GeSe crystal 700 to prevent oxidation and degradation of the GeSe crystal 700. The passivation layer 800 may be formed using materials such as silicon dioxide (SiO₂), aluminum oxide (Al₂O₃), or other chemically stable dielectrics that form a conformal coating over the surface of the GeSe crystal 700 without significantly interfering with its optical properties. The deposited passivation layer 800 inhibits exposure of the GeSe crystal 700 to environmental factors such as oxygen, humidity, or other reactive species that may otherwise deteriorate its structural integrity or optical performance over time. The passivation layer 800 allows long-term stability and operational reliability of the polarization modulator 108 by preserving the birefringence characteristics of the GeSe crystal under continuous usage. In addition, the passivation enhances compatibility with encapsulation and packaging techniques required for integration of the polarization modulator 108 within the housing 102 of the system 100.

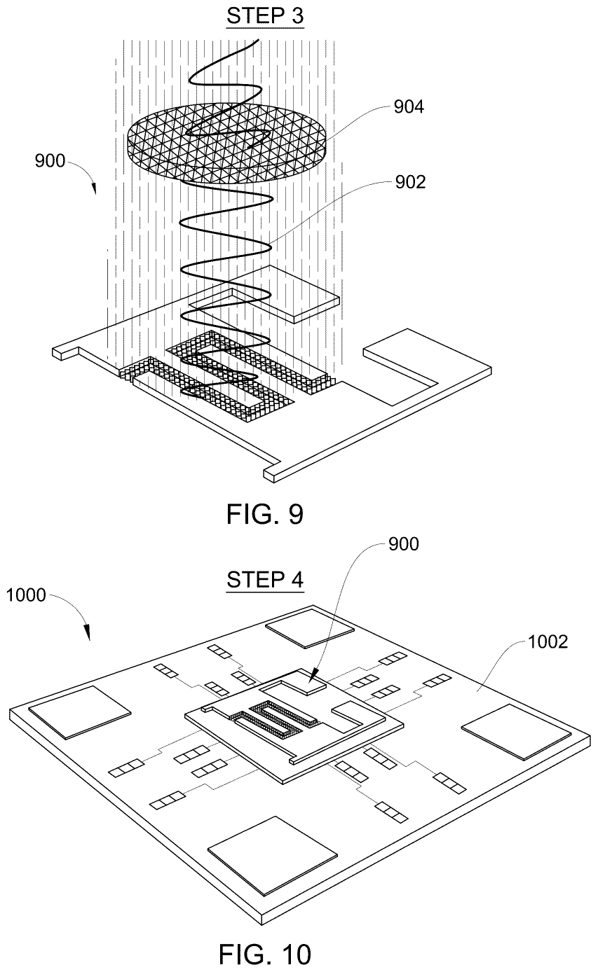

FIG. 9 shows the polarization modulator 900 (such as the polarization modulator 108) ready for testing, in accordance with an embodiment of the present disclosure. The testing comprises Step 3 of the process. Subsequently, the polarization modulator 900 is tested by subjecting polarization modulator 900 to polarized modulated light 902 under controlled laboratory conditions. A test sample 904 is arranged in the optical path of the polarized modulated light 902 to perform the testing.

During the testing process, linearly polarized light 902 with a predefined polarization state is directed toward the polarization modulator 900. The modulator is evaluated based on its ability to induce measurable changes in the polarization state of the linearly polarized light 902 in response to simulated biological interactions via transmission of the linearly polarized light 902 through the test sample 904. The resulting modulated light is analyzed using polarization-resolving equipment to verify the accuracy, consistency, and sensitivity of the polarization modulator 900.

The test results are used to assess the performance of the polarization modulator 900, including its modulation depth, alignment stability, and ability to operate under varying environmental conditions. This ensures the polarization modulator 900 meets the required operational standards for integration within the non-invasive health monitoring device.

FIG. 10 shows the tested polarization modulator 900 of FIG. 9 integrated into a processor 1000 (such as the processor 112 of FIGS. 1-3) to obtain an integrated circuit (IC) 1002, in accordance with an embodiment of the present disclosure. The IC 1000 combines the functionalities of the polarization modulator 900 and the processor 1002, enabling streamlined processing of polarization-modulated light for accurate determination of target analyte concentrations.

In certain implementations, the IC 1000 optionally includes functionalities of a photodetector (such as the photodetector 110 of FIGS. 1-3), facilitating direct detection of modulated light without requiring additional discrete components. This integration allows for efficient signal acquisition, modulation, and processing within a single compact module. The consolidation of these functionalities enables the IC 1000 to reduce overall complexity of the non-invasive health monitoring device (such as the non-invasive health monitoring device 100 of FIGS. 1-3), minimizeslatency in signal processing, and support seamless integration into portable and wearable non-invasive health monitoring devices.

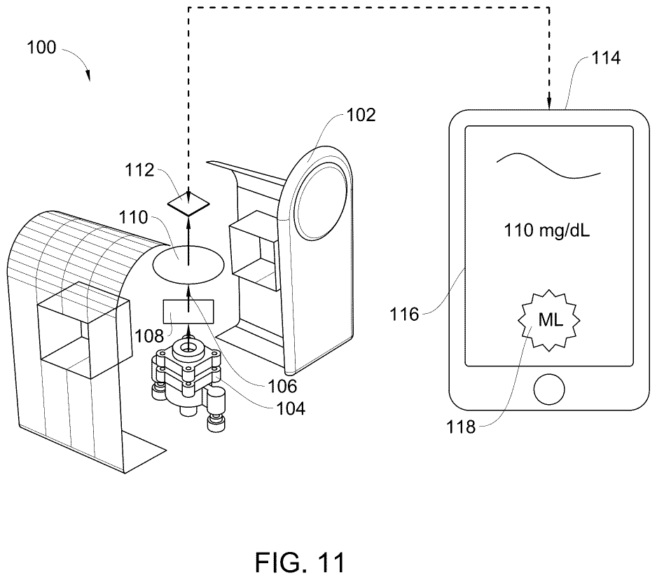

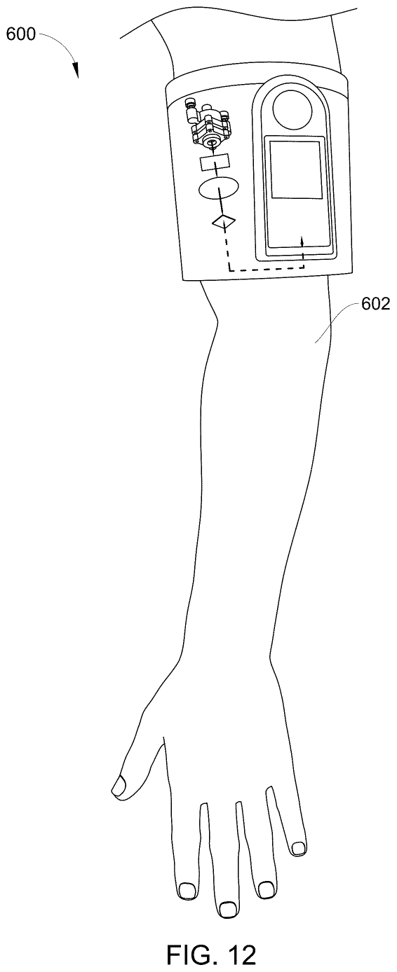

Referring to FIG. 11, there is shown an implementation of the non-invasive health monitoring device 100 of FIG. 1 with a companion app for a smartphone 114, in accordance with an embodiment of the present disclosure. The smartphone 114 features a smartphone user interface (UI) 116 displaying the companion app, which is configured to present real-time measurements indicative of target analyte concentrations, as determined by the non-invasive health monitoring device 100.

The companion app incorporates an integrated machine learning (ML) module 118, which processes historical data and patterns received from the non-invasive health monitoring device 100 to enhance measurement accuracy and provide personalized health insights. The ML module 118 enables adaptive calibration of the non-invasive health monitoring device 100 based on user-specific physiological parameters, thereby improving long-term reliability and reducing measurement variability.

This implementation facilitates seamless data synchronization between the non-invasive health monitoring device 100 and the smartphone 114, allowing users to access detailed trends, alerts, and recommendations via the companion app. Such integration supports proactive health monitoring and the potential for remote health management through communication with external healthcare systems.

Referring to FIG. 12, there is shown a three-dimensional view of an upper-arm wearable armband for the non-invasive health monitoring device, in accordance with an embodiment of the present disclosure. The armband comprises a transparent housing shell 120 that encases and protects internal components while providing a lightweight and ergonomic design for comfortable upper-arm wear. The housing shell 120 allows visibility into the internal assembly, highlighting the sensor module block 122, which houses the optical and electronic components essential for non-invasive analyte measurement.

The armband is secured around the user's upper arm via a band strap 124, which may be implemented using adjustable, skin-friendly materials to ensure a snug and stable fit. The integrated display 126 is positioned on the outer surface of the armband, enabling users to view real-time measurements and alerts directly on the device without requiring a companion app or external device.

A thin arrow depicted in FIG. 12 illustrates the optical path, indicating the transmission of light from the sensor module block 122 into the tissue for analyte detection. The compact design of the armband, combined with its integrated display and secure strap, facilitates continuous real-time monitoring of target analytes while ensuring ease of use and portability. This configuration enhances the applicability of the device for ambulatory and active users, supporting a wide range of health monitoring scenarios.

In certain embodiments (such as the non-invasive health monitoring devices 400 of FIGS. 4-5 and the non-invasive health monitoring device 600 of FIG. 6), the device further comprises an integrated display to present the measurement indicative of the target analyte concentration. The integrated display may be implemented as a digital screen such as a liquid crystal display (LCD), organic light-emitting diode (OLED) display, or electronic paper display configured to render numerical or graphical representations of the detected concentration level.

The integrated display provides real-time visual feedback to the user, allowing immediate awareness of physiological status without requiring external monitoring equipment. The integration of the display within the non-invasive health monitoring device enables compact, self-contained operation and facilitates portability and ease of use in home, clinical, or ambulatory settings. In addition, the display may support user interface functions for viewing historical data, setting thresholds, or receiving alerts based on analyte trends, such as, through implementation of the display as a touchscreen.

In certain embodiments (such as the non-invasive health monitoring devices 100 of FIGS. 1-3, non-invasive health monitoring devices 400 of FIGS. 4-5 and the non-invasive health monitoring device 600 of FIG. 6), the device further comprises a wireless communication module connected to the processor 112. The wireless communication module is configured to transmit the measurement indicative of the target analyte concentration to an external computing device. The term “wireless communication module” as used throughout the disclosure relates to an embedded communication subsystem configured to enable data transmission using wireless protocols such as Bluetooth, Wi-Fi, Zigbee, or cellular communication standards. The wireless communication module facilitates seamless integration of the device with external systems for extended monitoring and data management.

The external computing device may be implemented as a smartphone, tablet, or laptop configured to execute a corresponding application for displaying, analyzing, or archiving the measurement. In clinical or institutional settings, the external computing device may further include an electronic medical record (EMR) system, patient data management system (PDMS), or centralized health information infrastructure maintained by healthcare providers. The wireless communication module enables remote patient monitoring, longitudinal health trend analysis, and real-time clinical interventions based on transmitted analyte concentration data.

The present disclosure also provides a method for non-invasive analyte measurement, the comprising emitting linearly polarized light, transmitting the light through a body part, modulating the light via a GeSe crystal, detecting a polarization state change in the modulated light and determining a target analyte concentration based on the detected change.

Additionally, the present disclosure provides a non-transitory computer-readable medium storing instructions that, when executed by a processor, cause the processor to perform the aforementioned method.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments, those skilled in the art will recognize that the embodiments herein can be practiced with modification within the spirit and scope of the embodiments as described herein.

### Definitions

It will be appreciated that various aspects of the invention may be embodied as a method, system, computer readable medium, and/or computer program product. Aspects of the invention may take the form of hardware embodiments, software embodiments (including firmware, resident software, micro-code, etc.), or embodiments combining software and hardware aspects that may all generally be referred to herein as a “circuit,” “module,” or “system.” Furthermore, the methods of the invention may take the form of a computer program product on a computer-usable storage medium having computer-usable program code embodied in the medium.

Any suitable computer useable medium may be utilized for software aspects of the invention. The computer-usable or computer-readable medium may be, for example but not limited to, an electronic, magnetic, optical, electromagnetic, infrared, or semiconductor system, apparatus, device, or propagation medium. The computer readable medium may include transitory and/or non-transitory embodiments. More specific embodiments (a non-exhaustive list) of the computer-readable medium would include some or all of the following:
an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission medium such as those supporting the Internet or an intranet, or a magnetic storage device. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device.

Program code for carrying out operations of the invention may be written in an object-oriented programming language such as Java, Smalltalk, C++ or the like. However, the program code for carrying out operations of the invention may also be written in conventional procedural programming languages, such as the “C” programming language or similar programming languages. The program code may be executed by a processor, application specific integrated circuit (ASIC), or other component that executes the program code. The program code may be simply referred to as a software application that is stored in memory (such as the computer readable medium discussed above). The program code may cause the processor (or any processor-controlled device) to produce a graphical user interface (“GUI”). The graphical user interface may be visually produced on a display device, yet the graphical user interface may also have audible features. The program code, however, may operate in any processor-controlled device, such as a computer, server, personal digital assistant, phone, television, or any processor-controlled device utilizing the processor and/or a digital signal processor.

The program code may locally and/or remotely execute. The program code, for example, may be entirely or partially stored in local memory of the processor-controlled device. The program code, however, may also be at least partially remotely stored, accessed, and downloaded to the processor-controlled device. A user's computer, for example, may entirely execute the program code or only partly execute the program code. The program code may be a stand-alone software package that is at least partly on the user's computer and/or partly executed on a remote computer or entirely on a remote computer or server. In the latter scenario, the remote computer may be connected to the user's computer through a communications network.

The invention may be applied regardless of networking environment. The communications network may be a cable network operating in the radio-frequency domain and/or the Internet Protocol (IP) domain. The communications network, however, may also include a distributed computing network, such as the Internet (sometimes alternatively known as the “World Wide Web”), an intranet, a local-area network (LAN), and/or a wide-area network (WAN). The communications network may include coaxial cables, copper wires, fiber optic lines, and/or hybrid-coaxial lines. The communications network includes wireless portions utilizing any portion of the electromagnetic spectrum and any signaling standard (such as the IEEE 802 family of standards, GSM/CDMA/TDMA or any cellular standard, and/or the ISM band). The communications network may even include powerline portions, in which signals are communicated via electrical wiring. The invention may be applied to any wireless/wireline communications network, regardless of physical componentry, physical configuration, or communications standard(s).

In some aspects, wireless communication interfaces may include, but are not limited to, an Intranet connection, Internet, Personal Area Networks (PANs) for the exchange of data over short distances, e.g., using short-wavelength radio transmissions in the industrial, scientific, and medical (ISM) band ISM band from 2400-2480 MHz) from fixed and mobile devices (e.g., Bluetooth® technology), wireless fidelity (Wi-Fi), Wi-Max, IEEE 802.1 1 technology, radio frequency (RF), Infrared Data Association (IrDA) compatible protocols, Local Area Networks (LANs), Wide Area Networks (WANs), Shared Wireless Access Protocol (SWAP), Zigbee, Near-Field Communication (NFC), LiFi, 5G, any combinations thereof, and other types of wireless networking protocols.

Certain aspects of invention are described with reference to various methods and method steps. It will be understood that each method step can be implemented by the program code and/or by machine instructions. The program code and/or the machine instructions may create means for implementing the functions/acts specified in the methods.

The program code may also be stored in a computer-readable memory that can direct the processor, computer, or other programmable data processing apparatus to function in a particular manner, such that the program code stored in the computer-readable memory produce or transform an article of manufacture including instruction means which implement various aspects of the method steps.

The program code may also be loaded onto a computer or other programmable data processing apparatus to cause a series of operational steps to be performed to produce a processor/computer implemented process such that the program code provides steps for implementing various functions/acts specified in the methods of the invention.

Any of a variety of light sources may be used to provide the excitation and/or imaging light, including but not limited to, tungsten lamps, tungsten-halogen lamps, arc lamps, lasers, light emitting diodes (LEDs), or laser diodes.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. Although the foregoing subject matter has been described in some detail by way of illustration and example for purposes of clarity of understanding, it will be understood by those skilled in the art that certain changes and modifications can be practiced within the scope of the appended claims.

What is claimed:

1. A non-invasive health monitoring device, the device comprising:
a housing configured to receive a body part of a user;
a light emitter disposed within the housing, wherein the light emitter is configured to emit linearly polarized light and wherein the emitted linearly polarized light is transmitted through the body part of the user;
a polarization modulator disposed within the housing on an optical path of the light emitter, wherein the polarization modulator comprises a germanium-selenium (GeSe) crystal configured to modulate a polarization state of transmitted light after interaction of the emitted linearly polarized light with the body part of the user;
a photodetector disposed within the housing on an optical path of the polarization modulator, wherein the photodetector is configured to receive modulated light after modulation of the light by the polarization modulator and wherein the photodetector is configured to detect a polarization state of the modulated light;
a processor communicatively coupled to the photodetector, wherein the processor is configured to:
analyze changes in the polarization state of the modulated light with respect to a polarization state of the emitted linearly polarized light to determine a target analyte concentration within the body part of the user; and
output a corresponding measurement indicative of the target analyte concentration.

2. The device of claim 1, wherein the non-invasive health monitoring is a glucometer and wherein the target analyte concentration is blood glucose concentration.

3. The device of claim 1, further comprising an optical sensing chamber, wherein the non-invasive health monitoring device is implemented as a finger-insert device configured to receive a finger of the user into the optical sensing chamber for monitoring the target analyte concentration on demand.

4. The device of claim 1, wherein the non-invasive health monitoring device is implemented as a wrist-wearable device configured to receive a wrist of the user for continuous real-time monitoring of the target analyte concentration.

5. The device of claim 1, wherein the processor is configured to execute an artificial intelligence (AI) algorithm for processing variations in the polarization states of the modulated light to improve accuracy of measurement of target analyte concentrations.

6. The device of claim 1, wherein the GeSe crystal is processed using mechanical exfoliation to achieve a uniform nanoscale thickness.

7. The device of claim 1, wherein the GeSe crystal is subjected to thermal treatment at temperatures between 300° C. and 500° C. to enhance birefringence properties of the GeSe crystal.

8. The device of claim 1, wherein a surface passivation layer is deposited on the GeSe crystal to prevent oxidation and degradation of the GeSe crystal.

9. The device of claim 1, further comprising an integrated display to present the measurement indicative of the target analyte concentration.

10. The device of claim 1, further comprising a wireless communication module connected to the processor, wherein wireless communication module is configured to transmit the measurement indicative of the target analyte concentration to an external computing device.

11. A method for non-invasive analyte measurement, comprising:
(a) emitting linearly polarized light;
(b) transmitting the light through a body part;
(c) modulating the light via a GeSe crystal;
(d) detecting a polarization state change in the modulated light; and
(e) determining a target analyte concentration based on the detected change.

12. A non-transitory computer-readable medium storing instructions that, when executed by a processor, cause the processor to perform the method of claim 11.
